(19) United States
(12) Patent Application Publication
(10) Pub. No.: US 2008/0020387 A1
(43) Pub. Date: Jan. 24, 2008

(54) BIOMARKER-OPTIMIZED ADHD TREATMENT

(76) Inventor: Donald Gilbert Lawrence, Cincinnati, OH (US)

Correspondence Address: ELI LILLY & COMPANY, PATENT DIVISION, P.O. BOX 6288, INDIANAPOLIS, IN, 46206-6288, US

(21) Appl. No.: 11/694,099

(22) Filed: Mar. 30, 2007

Related U.S. Application Data
(60) Provisional application No. 60/788,008, filed on Mar. 31, 2006.

(51) Int. Cl. C12Q 1/68; G01N 33/48

(52) U.S. Cl. 435/6; 436/94

(57) ABSTRACT

Methods of predicting patient responsiveness to treatment of attention-deficit/hyperactivity disorder (ADHD) with selective norepinephrine reuptake inhibitors; identifying individuals requiring a higher than normal dose of atomoxetine for treating ADHD; and predicting patient responsiveness to treatment of neuropsychiatric diseases or disorders responsive to treatment with selective norepinephrine reuptake inhibitors are provided. These methods are based on the identification of the variable number of tandem repeats (VNTR) polymorphism present in the 3′-untranslated region of the human dopamine transporter 1 (DAT 1) gene present in patient body fluid or tissue samples. Patients with a 10/10 VNTR genotype are considered poor responders to treatment with atomoxetine and other selective norepinephrine reuptake inhibitors for the indicated conditions.

This application claims the benefit of priority of U.S. Provisional Application No. 60/788008, filed Mar. 31, 2006, the contents of which are herein incorporated by reference in their entirety.

The present invention relates to a biomarker predictive of patient responsiveness to atomoxetine and other selective norepinephrine reuptake inhibitors for the treatment of Attention-Deficit/Hyperactivity Disorder (ADHD) and other neuropsychiatric disorders amenable to treatment with these drugs.

The use of atomoxetine hydrochloride, a selective norepinephrine reuptake inhibitor, as an agent for the treatment of ADHD in children, adolescents, and adults is disclosed in U.S. Pat. No. 5,658,590. This compound was first approved for commercial use by the U.S. Food and Drug Administration in November, 2002. Subsequent approvals have been granted in a number of European countries and elsewhere. The pharmaceutical product is currently marketed for this purpose by Eli Lilly and Company under the name Strattera®.

As is the case with many other drugs, variation in patient response to atomoxetine is a significant therapeutic issue: clinical evidence suggests that approximately 40% of ADHD patients do not respond to atomoxetine treatment for ADHD (Michelson et al. (2002)Am. J. Psychiatry 159(11):1896-1901). A similar non-response rate is seen for ADHD treatment with methylphenidate.

In view of the demonstrated effectiveness of atomoxetine in reducing symptoms of ADHD in most patients, it has become important for physicians to have objective and reliable means to predict the likelihood of success of treatment in individual situations. There is a fundamental need to be able to predict patient responses to this drug, which would facilitate proper patient selection, optimized dosing regimens, reduced adverse events, and improved clinical outcomes. An objective test employing molecular biological data to aid clinicians in making treatment choices for ADHD patients would be helpful.

The present invention provides a solution to the problem of identifying ADHD patients, or individuals susceptible to developing ADHD, most likely to benefit from treatment with atomoxetine, or a pharmaceutically acceptable salt thereof, by providing genetic, clinical, neurophysiological, and multiple treatment-response biomarkers and methods for predicting patient responsiveness to this drug. Such biomarkers can be used to identify atomoxetine responders from non-responders in a patient population. Specifically, the present inventor has found that variations in the number of 40-basepair (bp) tandem repeats (Variable Number of Tandem Repeats or VNTR) polymorphism in the 3′-untranslated region (UTR) of the human Dopamine Transporter Gene 1 (DAT1 gene; SLC6A3 gene) locus (“the DAT1 gene VNTR polymorphism”), first identified by Vandenbergh et al. ((1992) Genomics 14(4):1104-1106), can be used to predict patient response to atomoxetine for the treatment of ADHD. The data disclosed below describe genotyping of the DAT1 gene in ADHD patients to determine the identity, i.e., number, of DAT1 40-basepair tandem repeats, detailed symptom severity assessment, and transcranial magnetic stimulation (TMS) of motor cortex to measure neurophysiological changes induced by atomoxetine. The relationship between DAT1 genotypes and neurophysiological responses to atomoxetine disclosed herein has not previously been reported.

Accordingly, in a first aspect, the present invention provides a method of predicting responsiveness to treatment of attention-deficit/hyperactivity disorder in a human with a compound selected from the group consisting of atomoxetine, a compound of formula I:

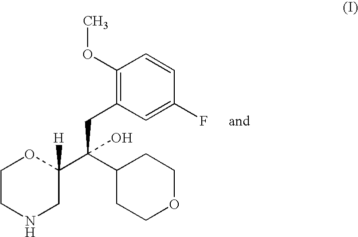

reboxetine, or

a pharmaceutically acceptable salt thereof, comprising:

(a) obtaining a sample of body fluid or other tissue from said human, and

(b) determining the identity of the variable number of tandem repeats (VNTR) polymorphism present in the 3′-untranslated region of each of the two DAT1 genes present in said human's sample,

wherein if one copy of said DAT1 gene has nine or fewer repeat alleles in said VNTR polymorphism and the other copy has ten or more repeat alleles in said VNTR polymorphism, or if both copies of said DAT 1 gene have nine or fewer repeat alleles in said VNTR polymorphism, then said human is predicted to be a good responder to treatment with said compound or pharmaceutically acceptable salt thereof for attention-deficit/hyperactivity disorder, and

wherein if both copies of said DAT1 gene have ten or more repeat alleles in said VNTR polymorphism, then said human is predicted to be a poor responder to treatment with said compound or pharmaceutically acceptable salt thereof for attention-deficit/hyperactivity disorder.

In another aspect, the present invention provides a method of identifying a human requiring a higher than normal dose of atomoxetine or a pharmaceutically acceptable salt thereof for the treatment of attention-deficit/hyperactivity disorder, comprising:

(a) obtaining a sample of body fluid or other tissue from a human suffering from, or susceptible to suffering from, attention-deficit/hyperactivity disorder; and

(b) determining the identity of the variable number of tandem repeats (VNTR) polymorphism present in the 3′-untranslated region of each of the two DAT1 genes present in said human's sample,

wherein if both copies of said DAT1 gene have ten or more repeat alleles in said VNTR polymorphism, then said human is identified as one requiring a higher than normal dose of atomoxetine or a pharmaceutically acceptable salt thereof for the treatment of attention-deficit/hyperactivity disorder,

wherein a normal dose for a child or adolescent up to 70 kg body weight is in the range of from about 1.2 mg/kg body weight to about 1.4 mg/kg bodyweight, or 100 mg, whichever is less,

wherein a normal dose for a child or adolescent over 70 kg body weight is in the range of from about 80 mg/day to about 100 mg/day, and

wherein a normal dose for an adult is about 100 mg/day.

In another aspect, the present invention provides a method of predicting responsiveness to treatment of a neuropsychiatric disease or disorder responsive to treatment with a selective norepinephrine reuptake inhibitor in a human with a compound selected from the group consisting of atomoxetine, a compound of formula I:

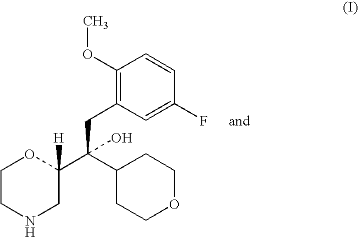

reboxetine, or

a pharmaceutically acceptable salt thereof, comprising:

(a) obtaining a sample of body fluid or other tissue from said human, and

(b) determining the identity of the variable number of tandem repeats (VNTR) polymorphism present in the 3′-untranslated region of each of the two DAT1 genes present in said human's sample,

wherein if one copy of said DAT1 gene has nine or fewer repeat alleles in said VNTR polymorphism and the other copy has ten or more repeat alleles in said VNTR polymorphism, or if both copies of said DAT1 gene have nine or fewer repeat alleles in said VNTR polymorphism, then said human is predicted to be a good responder to treatment with said compound or pharmaceutically acceptable salt thereof for said neuropsychiatric disease or disorder, and

wherein if both copies of said DAT1 gene have ten or more repeat alleles in said VNTR polymorphism, then said human is predicted to be a poor responder to treatment with said compound or pharmaceutically acceptable salt thereof for said neuropsychiatric disease or disorder.

Such neuropsychiatric diseases or disorders include, for example, cognitive impairment or failure; tics or Tourette's disorder; a pervasive developmental disorder such as autism, Rett's disease, Asperger's disease, etc.; conduct disorder; oppositional defiant disorder; a learning disability or motor skills disorder such as reading disorder (dyslexia); etc., as disclosed in the Diagnostic and Statistical Manual of Mental Disorders, Fourth Edition (2000), Text Revision (DSM-IV-TR™), American Psychiatric Association, Washington, D.C.

In any of the foregoing methods, atomoxetine or the compound of formula I can be in the form of a hydrochloride salt, and the body fluid or other tissue can be blood, semen, saliva, tears, urine, fecal material, sweat, a buccal smear, skin, hair, or biopsies of specific organ tissues, such as muscle or nerve tissue.

In addition to atomoxetine and the compound of formula I, other selective norepinephrine reuptake inhibitors to which the methods and kits described herein can be applied include, but are not limited to, reboxetine (Edronax®), 2-[α-(2-ethoxy)phenoxybenzyl]morpholine, which is usually administered as the racemate. This was first taught by U.S. Pat. No. 4,229,449, which describes its utility for the treatment of depression. As used herein, the term “reboxetine” refers to any acid addition salt or the free base of the molecule existing as the racemate, either enantiomer, or any diastereomer, e.g., S,S-reboxetine.

Further scope of the applicability of the present invention will become apparent from the detailed description and examples provided below, which are given for the purpose of non-limiting illustration only.

The present invention encompasses measuring the effects of pharmacologically relevant doses of atomoxetine on short interval cortical inhibition (SICI) in youth affected with ADHD. SICI, measured in motor cortex with Transcranial Magnetic Stimulation (TMS), has been shown to be reduced in ADHD, and correlated with symptom severity. Supporting the SICI/ADHD relationship, in the only TMS study of the psychostimulant methylphenidate in children with ADHD, a single 10 mg dose was found to significantly increase (normalize) SICI (Moll et al. (2000) Neurosci. Lett. 284:121-125). However, no other laboratories have reproduced this result. To better understand mechanisms responsible for variable treatment responses, assessment is also made of responses to this drug in ADHD children in terms of the 10-repeat VNTR polymorphism present in the 3′-UTR of the DAT1 gene.

The identification of an association between a clinical response and a DAT1 gene 3′-UTR VNTR polymorphism is the basis for designing a diagnostic method to determine those individuals who will or will not respond to the treatment, or alternatively, who will respond at a lower level, and thus may require more intensive treatment, e.g., a higher dose of drug. DAT1 9 and 10 repeat VNTR alleles are most common. The diagnostic method can take the form of a direct DNA test, i.e., identifying 3′-UTR VNTR polymorphisms in the patient's DAT1 gene. Polymorphisms in this region may result in aberrant expression of the dopamine transporter, thereby affecting its function: nine or fewer repeats in the DAT1 gene have been correlated with poor expression.

In a preferred embodiment, this diagnostic method uses the predictive methods described herein, encompassing determining the genotype of a patient at the 3′-UTR of the DAT1 gene and using this information in a method of predicting the efficacy of atomoxetine in treating a patient suffering from ADHD, or at future risk of exhibiting ADHD symptoms. This comprises (a) obtaining a sample of body fluid, such as blood, or other tissue from the patient, and (b) determining the identity of the VNTR polymorphisms present in the two copies of the DAT1 gene in the patient's sample. On the basis of this information, the attending physician can determine whether an individual exhibiting ADHD symptoms, or prone to exhibiting such symptoms, is an appropriate candidate for treatment with atomoxetine.

The data disclosed herein measure and compare the effects of the ADHD medications atomoxetine and methylphenidate on SICI in ADHD children. In addition, because of the unexplained variation in prior studies of the effects of methylphenidate on SICI, the influence of the number of DAT1 10-repeat VNTR alleles on the neurophysiological effects of these medications is also determined. Patients with Tourette Syndrome (TS) in addition to ADHD are included because of the high rate of comorbidity between ADHD and tic disorder.

Conventional techniques well known in the art in molecular biology, microbiology, and recombinant DNA methodology can be used in practicing the present invention. Non-limiting examples of methods that can be used to genotype VNTR polymorphisms can be found in PCT International Publication WO 2005/118848 and Persico et al. (1995) Am. J. Psychiatry 152:134-136.

The methods of the present invention can be carried out by employing a kit for the identification of a patient's polymorphism pattern at the VNTR locus polymorphic site of the DAT1 gene, comprising a means for determining the genetic polymorphism pattern at the VNTR locus polymorphic site of this gene. This means can comprise oligo-nucleotides used to amplify the target region. Thus, the kit can contain, packaged in separate containers, a polymerase and a reaction buffer optimized for primer extension mediated by the polymerase, such as polymerase chain reaction (PCR). In addition to a means for determining a genetic polymorphism pattern at the VNTR locus polymorphic site of the DAT1 gene, this kit can also include means for collecting a body fluid sample, such as blood, and a means for obtaining genomic DNA from this blood for the analysis. Typically, the nucleic acid is isolated from a biological sample taken from the individual, such as a blood or tissue sample. Suitable tissue samples include whole blood, semen, saliva, tears, urine, fecal material, sweat, buccal smears, skin, and biopsies of specific organ tissues, such as muscle or nerve tissue, and hair. The kit can comprise a container suitable for containing the needed materials and a sample of body fluid from the said individual, and instructions for use of the kit. These instructions would include the proper use of the kit and the proper manor of interpreting the results, as well as suggestions for patient selection or management depending on the specifics of the individual tested with the kit.

The following example is given by way of illustration only, and is not to be construed as a limitation of this invention in any way.

### EXAMPLE 1

In order to deduce a correlation between clinical response to atomoxetine treatment for ADHD and a VNTR polymorphism in the 3′-UTR of the DAT1 gene, it is necessary to obtain data on the clinical responses exhibited by a population of individuals who receive treatment.

Accordingly, sixteen children with ADHD, aged 8-17, are studied. Seven are homozygotes for the 10-repeat allele. Subjects are given single oral doses of 0.5 mg/kg methylphenidate and 1.0 mg/kg atomoxetine at visits separated by one week in a randomized, double-blind crossover design. Paired and single TMS are used to measure SICI before and after drug administration. Medication and genotype effects on SICI are estimated with repeated measures, mixed model regression. The data indicate that medication effects differ significantly according to this DAT1 genotype (F_{2,13}=13.04, p=0.0008). Atomoxetine increases (improves) SICI in heterozygotes but not in 10-repeat homozygotes. Some variation in ADHD medication-induced neurophysiological responses is explained by differences in the number of 3′-UTR, 10-repeat VNTR alleles in DAT1.

Patients and Methods

Subject Recruitment, Diagnosis, Clinical Assessment

Sixteen children and adolescents with clinically diagnosed ADHD, some with comorbid TS, receiving no ADHD medications at the time of the visits, are recruited and are scheduled for two visits, separated by approximately one week. Confirmation of the clinical diagnoses is based on DSM-IV (Diagnostic and Statistical Manual of Mental Disorders, Fourth Edition (2000), Text Revision (DSM-IV-TRTM), published by the American Psychiatric Association, Washington, D.C.) criteria, and all available clinical information from the examiner and outside raters. Participation is timed such that any medicated patients are able to stop taking stimulant ADHD medications for at least 48 hours prior to the first study visits.

Study Design and Drug Administration

The study is a randomized, double-blind, crossover design comparing the effects of single doses of atomoxetine (ATX) and methylphenidate (MPH). Sixteen patients (age 8-17 years, mean age=12, ±3; 2 girls) participate. Nine have TS. Medication doses are clinically relevant: approximately 0.5 mg/kg for MPH and 1.0 mg/kg for ATX, administered as follows:

1) subjects 20-29 kg→10 mg MPH, 20 mg ATX; 2) subjects 30-49 kg→20 mg MPH, 40 mg ATX; 3) subjects 50 kg and above→30 mg MPH, 60 mg ATX. Subjects and investigators are blind to the order of treatment throughout the study. The one-week interval between study visits is based on published pharmacokinetic data for single doses of ATX.

Clinical Ratings

On the day of each study visit, prior to medication administration, ADHD severity is rated with direct parent interview with the ADHD Rating Scale, with a possible rating scale score of 0-54 (DuPaul et al. (1998) ADHD Rating Scale-IV: Checklist, norms, and clinical interpretations. New York: Guilford Press). These are performed without knowledge of treatment order, TMS, or genotyping results.

DAT1 Genotyping

Approximately 10 ml EDTA anticoagulated venous blood is obtained from each participant. Genomic DNA is isolated using the Promega Genomic DNA Purification kit (Promega Corporation, Madison, Wis.). DAT 3′ untranslated region (UTR) VNTR polymorphisms are genotyped with standard polymerase chain reaction (PCR) as described in Persico et al. ((1995) American Journal of Psychiatry 152: 134-6). Genotyping is performed blind to clinical rating and TMS results.

Neurophysiology

Neurophysiological studies are performed as described by Gilbert et al. ((2006) Neuropsychopharmacology 31 :442-449). TMS is performed by an investigator blind to genotype and clinical ratings.

Resting and active motor threshold (RMT and AMT) are measured using the method described in Mills and Nithi ((1997) Muscle and Nerve 20:570-576). SICI and intracortical facilitation (ICF) are measured with the Abductor Pollicis Brevis (APB) at rest, according to established paradigms (Kujirai et al. (1993) J. Physiol. 471:501-519; Ziemann et al. (1997) Electroencelphalogr. Clin. Neurophysiol. 105:430-437). SICI is measured with a conditioning pulse, 1% of stimulator output below AMT (˜70% of RMT), delivered 3 msec before a suprathreshold test pulse. ICF is measured with the conditioning pulse 10 msec before the test pulse. Twenty trials at each interval and 20 unconditioned test pulses are delivered in random order. SICI and ICF are expressed as the ratios of the mean motor evoked potential (MEP) amplitudes evoked by the pulse pairs divided by the mean amplitude of the MEP from the single test pulses.

After baseline TMS testing, subjects are administered study medication, and all measurements are repeated 90 minutes later, consistent with expected peak serum drug levels. Each TMS session takes approximately 30 minutes.

Statistical Analysis

All analyses are performed with SAS^{R} version 8.02 (The SAS Institute, Inc., Cary, N.C., USA) or SPSSR version 11.5.0 (Chicago, Ill.).

Primary analysis. The primary outcome of interest for this study is ADHD-medication, genotype, and medication*genotype effects on cortical inhibition (SICI). SICI measures are subjected to a mixed model, repeated measures analysis of variance using PROC MIXED. After assuring that baselines are not different and that there are no effects of order, we then model SICI=medication+DAT genotype+medication*genotype, combining baselines. Age and ADHD severity scores are also entered into the models as potential explanatory covariates. ICF is modeled in the same manner to determine if any effects are specific to cortical inhibition.

Secondary analyses. The secondary outcome of interest is to compare ATX and MPH effects on cortical inhibition (SICI). Two repeated measure regressions are performed: 1) the baselines-combined model above, and 2) a day-of-visit, baseline-adjusted model. Genotypes are entered to estimate genotype-specific effects of ATX vs. MPH. The Tic phenotype is entered to estimate TIC*medication and TIC*ATX vs. TIC*MPH effects. In addition, using Spearman, correlations of changes in SICI and ICF are assessed for ATX and MPH, and for SICI and ADHD severity.

Results

Demographics, Adverse Events, Dropouts

The median subject weight is 46 kg (26-80 kg). The median medication doses are 20 mg MPH and 40 mg ATX. No events require breaking the blind. Pre-treatment SICI correlates with the ADHD rating scale score (r=−0.68, p=0.005), with more severe symptoms correlating with larger ratios (less SICI).

Fifteen of the sixteen subjects attend both visits. Thus, data from 62 TMS sessions are analyzed. One parent's ADHD rating scale scores at visit one vs. visit two are highly inconsistent, which are judged unreliable, and are excluded from the covariate analysis.

Of 16 subjects, 10 experience no side effects, 6 experience one or more side effects on the day of one or both visits, and one reports several side effects by phone interview on the days after both visits. Reported side effects in 4 subjects after ATX/TMS are: numbness/tingling (1), loss of appetite (2), scalp pain (1), nausea (2), stomach pain (1), and headache (3). Reported side effects in 6 subjects after MPH/TMS are: headache (3), numbness/tingling (1), arm/other pain (2), abdominal pain (1), hearing change (1). All side effects are rated mild, except for one child who reports a moderate headache the day after MPH/TMS.

DAT1 Genotypes (Table 1)

Seven patients are homozygous for DAT VNTR 10/10 repeats, 8 heterozygous for 9/10 repeats, and one heterozygous for 8/10 repeats. Heterozygous subjects are combined into one group. Clinical features by DAT1 genotype are shown in Table 1.

TABLE 1
Clinical and Neurophysiological Characteristics by Genotype

| | DAT 9/10 | | DAT 10/10 | | |
|---|---|---|---|---|---|
| Clinical characteristics | (n = 9) | | (n = 7) | | p Value |
| Tics | 6 | | 3 | | 0.62 |
| Males | 8 | | 6 | | 1.0 |
| Caucasian | 8 | | 4 | | 0.26 |
| African American | 1 | | 3 | | — |
| Age mean (SD) | 12.9 | (2.2) | 11.6 | (3.5) | 0.38 |
| Inattention Score mean (SD) | 19 | (4.3) | 21 | (5.3) | 0.39 |
| Hyperactive/Impulsive Score | 14 | (8.9) | 18 | (4.9) | 0.31 |
| mean (SD) | | | | | |
| Total ADHDRS mean (SD) | 33 | (10.4) | 39 | (7.1) | 0.21 |
| Cortical Inhibition mean SICI | .50 | (.30) | .62 | (.17) | 0.46 |
| ratio (SD) | | | | | |
| Cortical Facilitation mean ICF | 1.15 | (.30) | 1.16 | (.15) | 0.92 |
| ratio (SD) | | | | | |

ADHDRS = Attention-deficit/hyperactivity Disorder Rating Scale. ICF = Intracortical facilitation. SICI = Short Interval Cortical Inhibition. SD = Standard Deviation.

There are no significant differences in any pre-treatment demographic, clinical, or neurophysiological measures between DAT1 9/10 and 10/10 genotypes, although at baseline, DAT 10/10 subjects have worse ADHD scores and less SICI.

Effects of DAT1 Genotype on Physiological Response to Medications (Table 2)

There is no effect of treatment order or difference between visit one versus visit two. There is no main effect of medication (F_{2,13}=0.51, p=0.61).

The effects of medication treatment on cortical inhibition differ with DAT1 genotype. In patients with a single 10-repeat DAT1 allele, both MPH and ATX tend to increase (normalize) SICI as evidenced by lower SICI ratios in these patients. In contrast, after medication, individuals homozygous for the DAT1 10-repeat allele have a slight decrease in SICI (an increase in MEP (SICI) amplitude ratios).

This DAT1 genotype by medication interaction is highly robust and significant in the baseline-combined (F_{2,13}=13.04, p=0.0008) analysis. DAT1 genotype is also highly significant in the day-of visit, baseline-adjusted analyses (F_{1,13}=9.31, p=0.0093), which is used to compare ATX and MPH effects. The interaction remains significant when the individual with the 8/10 genotype is excluded (data not shown). Baseline-adjusted estimates, by genotype, of post-MPH and post-ATX SICI are shown in Table 2.

TABLE 2
Post-MPH and Post-ATX Motor Cortex Inhibition

| | Cortical | | |
|---|---|---|---|
| | Inhibition | | |
| Effect | (SICI) | 95% CI | P value |
| Medication - ATX vs. MPH | | | 0.52 |
| ATX | 0.59 | 0.51-0.67 | |
| MPH | 0.54 | 0.46-0.63 | |
| DAT Genotype | | | 0.0086 |
| DAT 9, 10 | 0.50 | 0.43-0.56 | |
| DAT 10, 10 | 0.64 | 0.57-0.71 | |
| Medication * DAT Genotype | | | 0.89 |
| ATX and DAT 9, 10 | 0.51 | 0.40-0.62 | |
| ATX and DAT 10, 10 | 0.66 | 0.54-0.79 | |
| MPH and DAT 9, 10 | 0.47 | 0.36-0.59 | |
| MPH and DAT 10, 10 | 0.61 | 0.49-0.74 | |
| TIC Phenotype | | | 0.0056 |
| No Tics | 0.63 | 0.57-0.70 | |
| Tics | 0.50 | 0.44-0.56 | |
| Medication * TIC Phenotype | | | 0.26 |
| ATX and No Tics | 0.62 | 0.50-0.73 | |
| ATX and Tics | 0.56 | 0.45-0.66 | |
| MPH and No Tics | 0.64 | 0.52-0.76 | |
| MPH and Tics | 0.44 | 0.33-0.54 | |

Post-medication SICI, estimated after adjustment for the day-of-visit, pre-treatment SICI. Abbreviations: ATX = atomoxetine. DAT = dopamine transporter, 9 and 10 indicate numbers of tandem repeats (see text). MPH = methylphenidate. SICI = Short Interval Cortical Inhibition.

There are no main or interaction effects of medication and DAT genotype on ICF. Age and ADHD severity do not interact with the medication effects (data not shown).

There is also no specific interaction detected between ATX or MPH and DAT1. However, after ATX, MEP amplitude ratios at short (SICI inhibitory) and long (ICF facilitatory) intervals tend to either both increase or both decrease (r=0.52, p=0.048). MPH-induced changes in SICI and ICF are not correlated (r=0.032, p=0.91).

Effects of TS Phenotype on Physiological Response to Medications

The effects of medication on cortical SICI (F_{2,13}=7.20, p=0.0078) differ by TS phenotype. In patients with ADHD and TS, ATX and MPH increase (normalize) SICI as evidenced by lower SICI ratios in these patients. In contrast, in individuals with ADHD and no TS, ATX and MPH decrease SICI. Baseline-adjusted estimates, by TS phenotype, of post-MPH and post-ATX SICI are shown in Table 2. Post-treatment SICI is significantly greater in patients with TS. This SICI increase in comorbid patients is greatest after MPH, but there is no statistically significant interaction between TS diagnosis and medication type. Interaction effects between DAT1 and TS phenotypes cannot not be estimated due to sample size.

The data presented herein suggest that in children with ADHD, the number of DAT1 VNTR 10-repeat alleles influences the neurophysiological responses to ATX. Short interval cortical inhibition (SICI), a measure of cortical activity that is reduced in ADHD and correlates inversely with ADHD symptom severity, increases toward normal with medication in DAT1 9,10 heterozygotes, similar to changes in SICI previously reported in a German cohort of children with ADHD (Moll et al. (2000) Neurosci. Lett. 284: 121-125). This differs significantly from the effects in DAT1 10,10 homozygotes. When MPH and ATX increase SICI in ADHD children, as occurs in the heterozygotes, we expect that symptoms would be more likely to improve. In contrast, failure to increase SICI, as occurs in the DAT1 10/10 homozygotes, might be more likely to occur in clinical non-responders. Thus, the data are consistent with studies showing a poorer clinical response to MPH in individuals with the DAT1 10/10 genotype (e.g., Cheon et al. (2005) European Neuropsycho-pharmacology 15:95-101) as well as divergent EEG responses. The relationship between DAT1 genotypes and neurophysiological responses to ATX disclosed herein has not previously been reported.

This is the first study to measure genotype effects on neurophysiologic responses to a norepinephrine reuptake inhibitor in children with ADHD. It is surprising and important that the DAT 1 receptor polymorphism modulates the effect of the selective norepinephrine reuptake inhibitor atomoxetine, and suggests that this polymorphism will be useful in optimizing the treatment of ADHD and other neuropsychiatric diseases or disorders amenable to treatment with this drug and, presumably, with other selective norepinephrine reuptake inhibitors as disclosed herein.

What is claimed is:

1. A method of predicting responsiveness to treatment of attention-deficit/hyperactivity disorder in a human with a compound selected from the group consisting of atomoxetine, a compound of formula I: 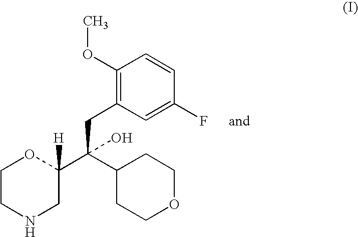
reboxetine, or
a pharmaceutically acceptable salt thereof, comprising:
(a) obtaining a sample of body fluid or other tissue from said human, and
(b) determining the identity of the variable number of tandem repeats (VNTR) polymorphism present in the 3′-untranslated region of each of the two DAT1 genes present in said human's sample,
wherein if one copy of said DAT1 gene has nine or fewer repeat alleles in said VNTR polymorphism and the other copy has ten or more repeat alleles in said VNTR polymorphism, or if both copies of said DAT1 gene have nine or fewer repeat alleles in said VNTR polymorphism, then said human is predicted to be a good responder to treatment with said compound or pharmaceutically acceptable salt thereof for attention-deficit/hyperactivity disorder, and
wherein if both copies of said DAT1 gene have ten or more repeat alleles in said VNTR polymorphism, then said human is predicted to be a poor responder to treatment with said compound or pharmaceutically acceptable salt thereof for attention-deficit/hyperactivity disorder.

2. The method of claim 1, wherein said compound is atomoxetine hydrochloride.

3. The method of claim 1, wherein said compound is a hydrochloride salt of said compound of formula I.

4. The method of claim 1, wherein said body fluid or other tissue is selected from the group consisting of blood, semen, saliva, tears, urine, fecal material, sweat, a buccal smear, skin, hair, and a biopsy of a specific organ tissue.

5. A method of identifying a human requiring a higher than normal dose of atomoxetine or a pharmaceutically acceptable salt thereof for the treatment of attention-deficit/hyperactivity disorder, comprising:
(a) obtaining a sample of body fluid or other tissue from a human suffering from, or susceptible to suffering from, attention-deficit/hyperactivity disorder; and
(b) determining the identity of the variable number of tandem repeats (VNTR) polymorphism present in the 3′-untranslated region of each of the two DAT1 genes present in said human's sample,
wherein if both copies of said DAT1 gene have ten or more repeat alleles in said VNTR polymorphism, then said human is identified as one requiring a higher than normal dose of atomoxetine or a pharmaceutically acceptable salt thereof for the treatment of attention-deficit/hyperactivity disorder,
wherein a normal dose for a child or adolescent up to 70 kg body weight is in the range of from about 1.2 mg/kg body weight to about 1.4 mg/kg bodyweight, or 100 mg, whichever is less,
wherein a normal dose for a child or adolescent over 70 kg body weight is in the range of from about 80 mg/day to about 100 mg/day, and
wherein a normal dose for an adult is about 100 mg/day.

6. The method of claim 5, wherein said pharmaceutically acceptable salt of atomoxetine is a hydrochloride salt.

7. The method of claim 5, wherein said body fluid or other tissue is selected from the group consisting of blood, semen, saliva, tears, urine, fecal material, sweat, a buccal smear, skin, hair, and a biopsy of a specific organ tissue.

8. A method of predicting responsiveness to treatment of a neuropsychiatric disease or disorder responsive to treatment with a selective norepinephrine reuptake inhibitor in a human with a compound selected from the group consisting of atomoxetine, a compound of formula I: 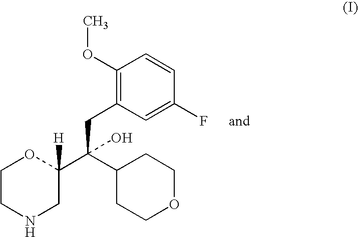
reboxetine, or
a pharmaceutically acceptable salt thereof, comprising:
(a) obtaining a sample of body fluid or other tissue from said human, and
(b) determining the identity of the variable number of tandem repeats (VNTR) polymorphism present in the 3′-untranslated region of each of the two DAT1 genes present in said human's sample,
wherein if one copy of said DAT1 gene has nine or fewer repeat alleles in said VNTR polymorphism and the other copy has ten or more repeat alleles in said VNTR polymorphism, or if both copies of said DAT1 gene have nine or fewer repeat alleles in said VNTR polymorphism, then said human is predicted to be a good responder to treatment with said compound or pharmaceutically acceptable salt thereof for said neuropsychiatric disease or disorder, and
wherein if both copies of said DAT1 gene have ten or more repeat alleles in said VNTR polymorphism, then said human is predicted to be a poor responder to treatment with said compound or pharmaceutically acceptable salt thereof for said neuropsychiatric disease or disorder.

9. The method of claim 8, wherein said neuropsychiatric disease or disorder responsive to treatment with a selective norepinephrine reuptake inhibitor is selected from the group consisting of cognitive impairment, cognitive failure, tics, Tourette's disorder, a pervasive developmental disorder, conduct disorder, oppositional defiant disorder, a learning disability, and motor skills disorder.

10. The method of claim 9, wherein said pervasive developmental disorder is selected from the group consisting of autism, Rett's disease, and Asperger's disease.

11. The method of claim 9, wherein said learning disability is reading disorder.

12. The method of claim 8, wherein said atomoxetine is in the form of a hydrochloride salt.

13. The method of claim 9, wherein said atomoxetine is in the form of a hydrochloride salt.

14. The method of claim 10, wherein said atomoxetine is in the form of a hydrochloride salt.

15. The method of claim 11, wherein said atomoxetine is in the form of a hydrochloride salt.

16. The method of claim 8, wherein said body fluid or other tissue is selected from the group consisting of blood, semen, saliva, tears, urine, fecal material, sweat, a buccal smear, skin, hair, and a biopsy of a specific organ tissue.

17. The method of claim 9, wherein said body fluid or other tissue is selected from the group consisting of blood, semen, saliva, tears, urine, fecal material, sweat, a buccal smear, skin, hair, and a biopsy of a specific organ tissue.

18. The method of claim 10, wherein said body fluid or other tissue is selected from the group consisting of blood, semen, saliva, tears, urine, fecal material, sweat, a buccal smear, skin, hair, and a biopsy of a specific organ tissue.

19. The method of claim 11, wherein said body fluid or other tissue is selected from the group consisting of blood, semen, saliva, tears, urine, fecal material, sweat, a buccal smear, skin, hair, and a biopsy of a specific organ tissue.
